(19) United States
(12) Patent Application Publication
(10) Pub. No.: US 2013/0123806 A1
(43) Pub. Date: May 16, 2013

(54) DEVICE FOR REMOVING ACROCHORDONS

(71) Applicants: Harold A. Howlett, Horn Lake, MS (US); John W. Davis, Munford, TN (US); Ronald M. Feret, Memphis, TN (US)

(72) Inventors: Harold A. Howlett, Horn Lake, MS (US); John W. Davis, Munford, TN (US); Ronald M. Feret, Memphis, TN (US)

(73) Assignee: MSD CONSUMER CARE, INC., Memphis, TN (US)

(21) Appl. No.: 13/293,902

(22) Filed: Nov. 10, 2011

(51) Int. Cl. A61B 17/50

(52) U.S. Cl. 606/131

(57) ABSTRACT

A device for occlusion of a skin tag projecting from a skin area includes a pressure layer having an upper side, an underside, and configured to enclose the skin tag and occlude blood flow to the enclosed skin tag, the pressure layer including a base member having an upper side, an underside, and configured to enclose the skin tag, at least one first planar spring connected to a first side of the base member at one end of the first planar spring, and a first occlusion member connected to an other end of the first planar spring for applying an occlusion force to the enclosed skin tag, and a top layer attached to the upper side of the pressure layer, the top layer covering at least a portion of the first planar spring, and the top layer having a hole configured to receive the enclosed skin tag.

### FIELD OF THE INVENTION

The present invention relates to a device for removing acrochordons, and a method of using the device for removing acrochordons.

### BACKGROUND

Common benign skin lesions may include verruca, seborrhoic keratoses, fibromas, histiocytomas (dermatofibromas), nevi and skin tags. A skin tag is a small outgrowth of epidermal and dermal tissue, one to several millimeters in size, usually flesh-colored and pedunculated. Skin tags display a variety of shapes, sizes and histology, as shown in FIG. 1, and are described by alternative names, of which acrochordon is the most accepted. The common sites for skin tags are the neck, axillae, trunk and groin, but they can occur all over the body.

The cellular characteristics of the skin tag may be important from the point of view of removal by pressure at the base. For example, for a floppy pedunculated skin tag in which the connective tissue stroma is thin and loose with few cells, externally exerted pressure would result in little resistance from the tissue, and the vasculature would easily collapse. These lesions tend to be smaller in size.

However, skin lesions in which the presence of nevus cells and a firm connective tissue network may protect the vasculature at the base from pressure may often be larger, broader in base and “dome shaped,” and exert more resistance to compression of the base.

A complete occlusion of the vasculature at the base of a skin tag could be expected to result in a necrosis of the skin tag. When occlusion of the base is incomplete, a degree of inflammation may be expected, which inflammatory response, by causing swelling, may enhance the occlusive effect.

The current medical treatment of skin tags involves clipping or shaving the lesion at the base, cryotherapy, or diathermy. Cryotherapy is perceived as inexpensive and does not require anesthesia, but there may be a number of drawbacks. For example, if cryotherapy were not targeted, it may result in damage to healthy tissue, it may be painful, and there may be a risk of blistering, scarring, and pigmentation changes. Alternatively, patients can undertake “self-treatment” by traumatizing the most accessible lesions in different ways, including tying off the lesions with a sewing thread or a rubber band such as, for example, in UK Patent Application No. GB 2 322 802. International Patent Publication Nos. WO 2006/067743 and WO 2011/092595 describe multiple embodiments of such “self-treatment” devices for occluding blood flow to skin tags.

Patients may be motivated to the removal of skin tags, but the health care infrastructure is often less interested because skin tags are benign and the cost of clinical intervention can be hard to justify, regardless of the psychological impact on the patient. In this regard, skin anomalies such as skin tags may strongly affect the patients' quality of life. For example, even slight changes in the skin may result in adjustments to social life, relationships, and even the ability to carry out daily tasks.

### SUMMARY

Accordingly, there is a need for a device for removing acrochordons safely, quickly and discreetly, which may be easily utilized by a patient with minimal pain, blistering, scarring, pigmentation changes or other potential drawbacks. Further, there is a need for a device that may be used by patients without significant professional medical intervention.

In a non-limiting embodiment of the present invention, a device for occlusion of a skin tag projecting from a skin area comprises a pressure layer having an upper side, an underside, and configured to enclose the skin tag and occlude blood flow to the enclosed skin tag, the pressure layer comprising a base member having an upper side, an underside, and configured to enclose the skin tag, at least one first planar spring connected to a first side of the base member at one end of the first planar spring, and a first occlusion member connected to an other end of the first planar spring for applying an occlusion force to the enclosed skin tag, and a top layer attached to the upper side of the pressure layer, the top layer covering at least a portion of the first planar spring, and the top layer having a hole configured to receive the enclosed skin tag.

In an alternative non-limiting embodiment of the invention, the pressure layer further comprises at least one second planar spring connected to a second side of the base member at one end of the second planar spring, and a second occlusion member connected to an other end of the second planar spring for applying the occlusion force to the enclosed skin tag.

In an alternative non-limiting embodiment of the invention, the underside of the pressure layer includes an adhesive for fixing the pressure layer to the skin area.

In an alternative non-limiting embodiment of the invention, the upper side of the pressure layer includes an adhesive for fixing the top layer to the pressure layer.

In an alternative non-limiting embodiment of the invention, the other end of the first planar spring faces the other end of the second planar spring.

In an alternative non-limiting embodiment of the invention, the first planar spring and the second planar spring are formed substantially in the shape of a “S”.

In an alternative non-limiting embodiment of the invention, a pair of first planar springs is connected to the first occlusion member.

In an alternative non-limiting embodiment of the invention, a pair of second planar springs is connected to the second occlusion member.

In an alternative non-limiting embodiment of the invention, the first occlusion member faces the second occlusion member for jointly applying the occlusion force to the enclosed skin tag.

In an alternative non-limiting embodiment of the invention, in a rest state, a gap is present between the first occlusion member and the second occlusion member.

In an alternative non-limiting embodiment of the invention, the gap is between approximately zero and approximately 20 mil.

In an alternative non-limiting embodiment of the invention, the device is one of flat and disc-shaped.

In an alternative non-limiting embodiment of the invention, the device is substantially square with rounded edges.

In an alternative non-limiting embodiment of the invention, the top layer is configured to at least one of select, center, and align the enclosed skin tag for occlusion.

In an alternative non-limiting embodiment of the invention, the first side and the second side of the base member of the pressure layer are configured to provide finger notches.

In an alternative non-limiting embodiment of the invention, the device further comprises a cover strip configured to cover at least one of the pressure layer, the top layer, and the enclosed skin tag.

In an alternative non-limiting embodiment of the invention, an underside of the cover strip includes an adhesive configured to adhere to at least one of the pressure layer, the top layer, the enclosed skin tag, and the skin area.

In an alternative non-limiting embodiment of the invention, the underside of the cover strip includes the adhesive along an entire length of the cover strip.

In an alternative non-limiting embodiment of the invention, the underside of the cover strip does not include adhesive in a central region of the cover strip configured to cover the enclosed skin tag.

In yet another non-limiting embodiment of the present invention, a method of using a device for occlusion of a skin tag projecting from a skin area, the device comprising a pressure layer having an upper side, an underside, and configured to enclose the skin tag and occlude blood flow to the enclosed skin tag, the pressure layer comprising a base member having an upper side, an underside, and configured to enclose the skin tag, at least one first planar spring connected to a first side of the base member at one end of the first planar spring, and a first occlusion member connected to an other end of the first planar spring, and a top layer attached to the upper side of the pressure layer, the top layer covering at least a portion of the first planar spring, and the top layer having a hole, comprises receiving the enclosed skin tag within the hole of the top layer.

In an alternative non-limiting embodiment of the invention, the pressure layer further comprises at least one second planar spring connected to a second side of the base member at one end of the second planar spring, and a second occlusion member connected to an other end of the second planar spring, and the method further comprises applying compression to the device, whereby a gap is formed between the first and second occlusion members, enclosing the skin tag within the gap formed between the first and second occlusion members, and releasing the compression on the device, whereby occlusion force is applied to the enclosed skin tag via the first and second occlusion members and the first and second planar springs.

In an alternative non-limiting embodiment of the invention, the compression is manually applied by a user's thumb and finger via finger notches provided at the first side and the second side of the base member of the pressure layer.

In an alternative non-limiting embodiment of the invention, the device is applied with one hand of a user.

In an alternative non-limiting embodiment of the invention, the method further comprises adhering the pressure layer to the skin area via an adhesive.

In an alternative non-limiting embodiment of the invention, the top layer at least one of selects, centers, and aligns the enclosed skin tag for occlusion.

In an alternative non-limiting embodiment of the invention, the method further comprises covering at least one of the pressure layer, the top layer, and the enclosed skin tag with a cover strip.

In an alternative non-limiting embodiment of the invention, the method further comprises adhering the cover strip to at least one of the pressure layer, the top layer, the enclosed skin tag, and the skin area via an adhesive.

In an alternative non-limiting embodiment of the invention, the covering includes covering the enclosed skin tag with the cover strip without adhering the cover strip to the enclosed skin tag.

Other features and aspects of the present invention will become more fully apparent from the following brief description of the drawings, the detailed description of the non-limiting embodiments, the appended claims and the accompanying drawings.

### BRIEF DESCRIPTION OF THE DRAWINGS

FIG. 1 illustrates a variety of types of skin tags.

FIG. 2 illustrates an exploded, perspective view of an exemplary embodiment of a device according to the present invention.

FIG. 3 illustrates a top plan view of a portion of an exemplary embodiment of a device according to the present invention.

FIG. 4 illustrates a top plan view of a portion of an exemplary embodiment of a device according to the present invention.

FIG. 5 illustrates a top plan view of a portion of an exemplary embodiment of a device according to the present invention.

FIG. 6 illustrates a top plan view of a portion of an exemplary embodiment of a device according to the present invention.

FIG. 7 illustrates a top plan view of a card including a plurality of an exemplary embodiment of a device according to the present invention.

FIG. 8 illustrates a top plan view of a cover strip for an exemplary embodiment of a device according to the present invention.

FIG. 9 illustrates a top plan view of a card including a plurality of cover strips for an exemplary embodiment of a device according to the present invention.

FIG. 10 illustrates a schematic flow diagram of a method of using a device according to the present invention.

### DETAILED DESCRIPTION OF THE EMBODIMENTS

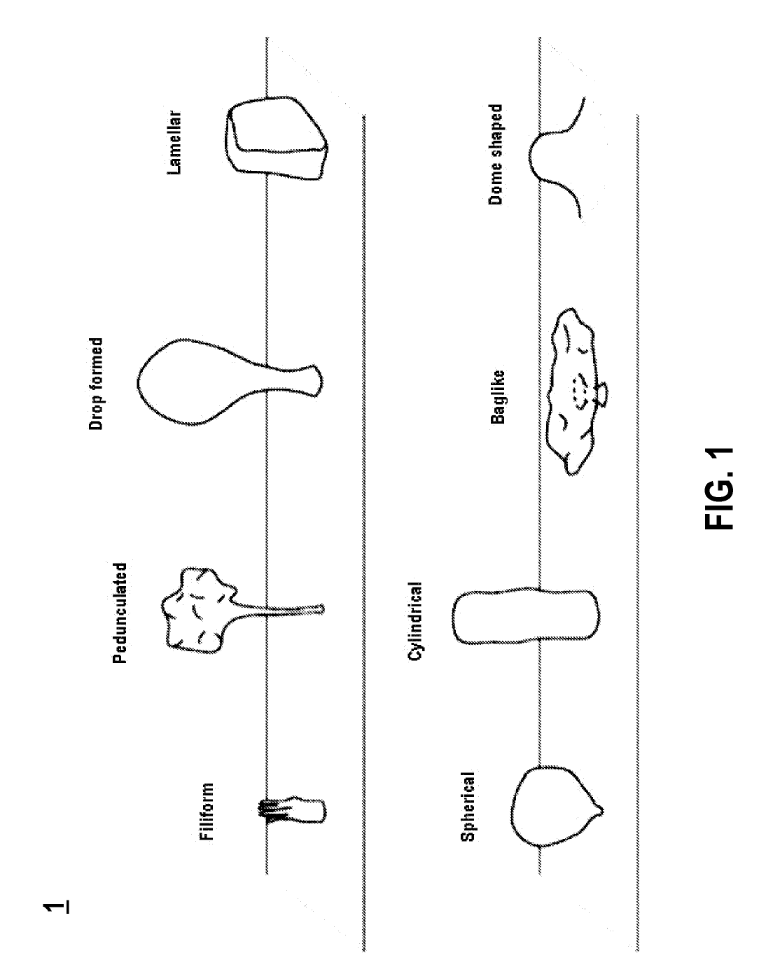

FIG. 1 illustrates a variety of types of skin tags, such as filiform, pedunculated, drop formed, lamellar, spherical, cylindrical, baglike, and dome shaped. Skin tags may exhibit a variety of shapes, sizes, histology, and other characteristics. The devices according to the present invention may be used to occlude, or occlusively remove, one or more of the variety of skin tags.

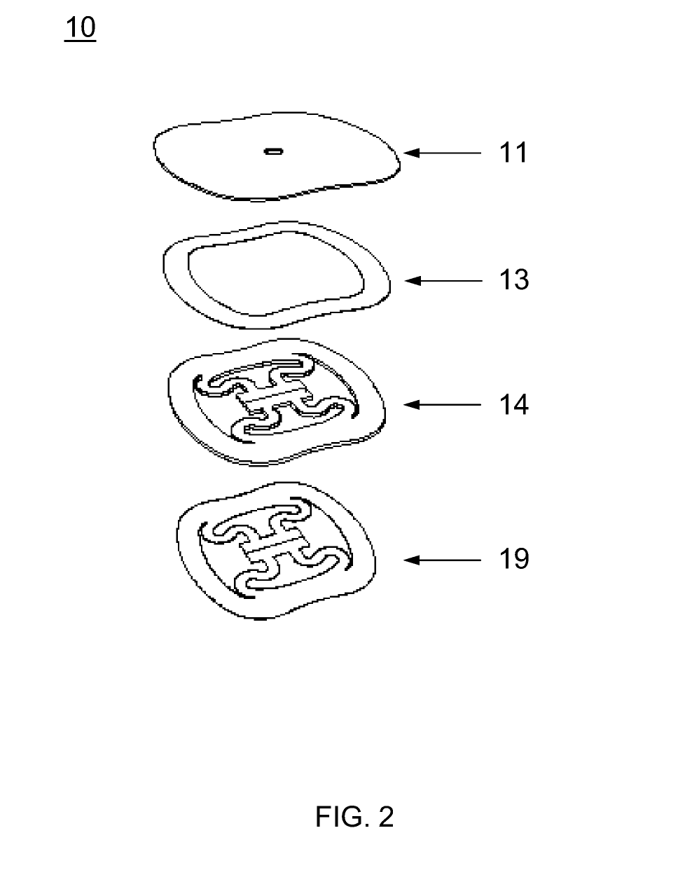

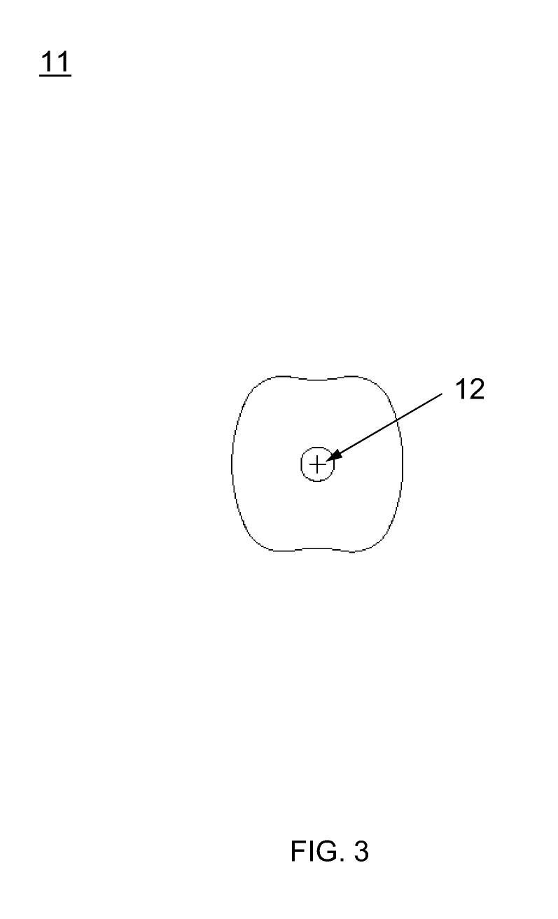

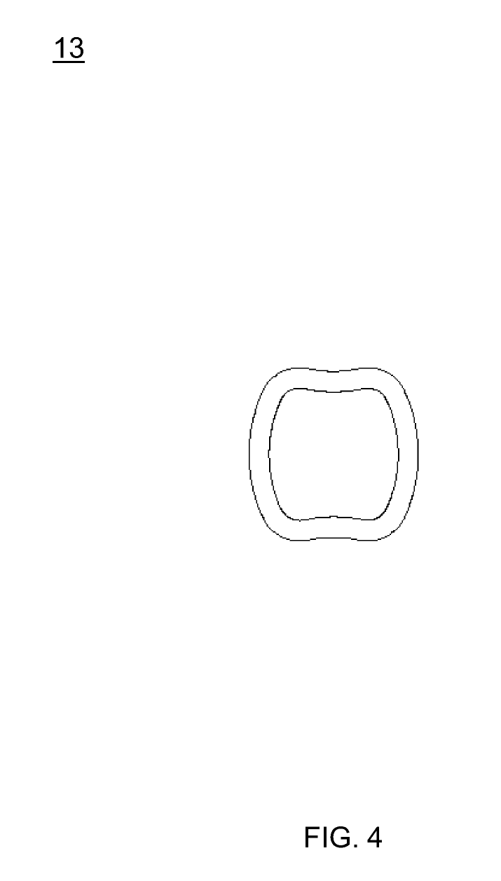

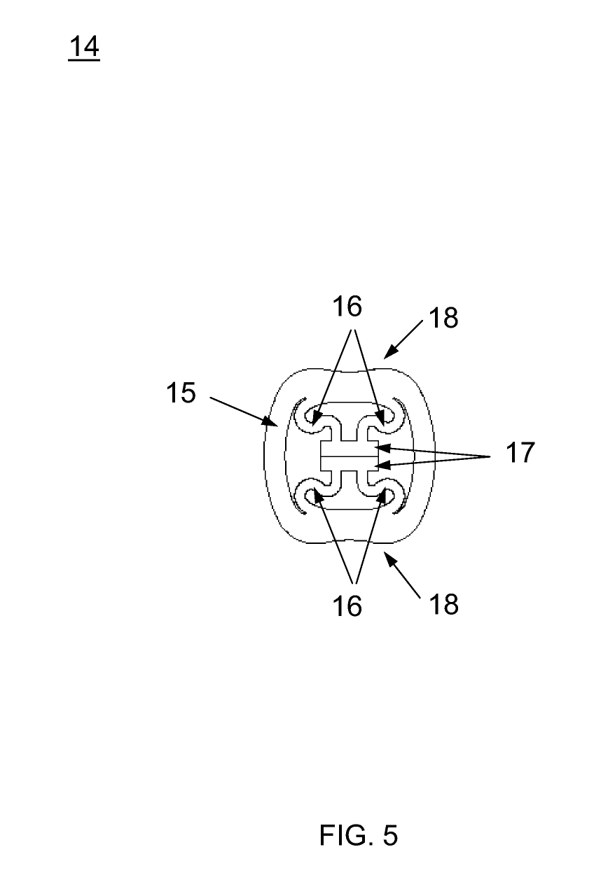

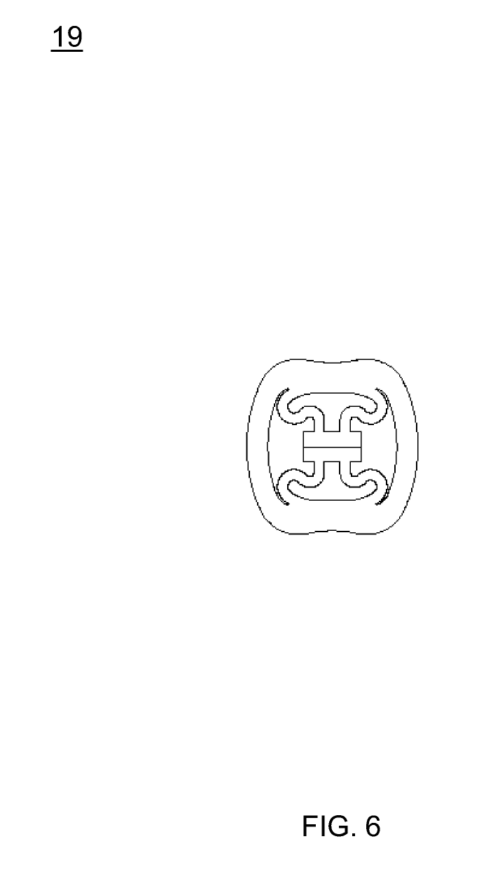

FIG. 2 illustrates an exploded, perspective view of an exemplary embodiment of a device 10 according to the present invention. The device 10 may be flat or disc-shaped. The device 10 includes a top layer 11, an intermediate adhesive layer 13, a pressure layer 14, and a base adhesive layer 19. FIG. 3 illustrates the top layer 11, FIG. 4 illustrates the intermediate adhesive layer 13, FIG. 5 illustrates the pressure layer 14, and FIG. 6 illustrates the base adhesive layer 19.

As shown in FIG. 5, the pressure layer 14 may include a base member 15, planar springs 16, and occlusion members 17. The base member 15 may be formed in a substantially square shape having rounded edges. Other shapes of the base member 15 may be possible, including rectangular, oval, circular, elliptical, polygonal, and others. In addition, the base member 15 may include finger notches 18 on opposing sides thereof, to facilitate gripping, alignment and overall use of the device 10.

Each planar spring 16 may be connected on one end to the base member 15, and connected on another end to an occlusion member 17. Each planar spring 16 may be formed substantially in a shape of an “S” and may be configured to provide occlusion pressure to an enclosed skin tag. As shown in FIG. 5, for example, a pair of planar springs 16 may be connected to each occlusion member 17. Alternatively, only one planar spring 16 or more than two planar springs 16 may be connected to each occlusion member 17.

Each occlusion member 17 may be connected to an end of a planar spring 16 remote from the base member 15. Each occlusion member 17 may be connected to one or more planar springs 16. The occlusion members 17 may contact one another and be pressed together by the occlusion pressure provided by the planar springs 16. As a result, blood flow to an enclosed skin tag situated between the occlusion members 17 may be occluded. Based on the shape, size, thickness, material properties, and other characteristics of the planar springs 16 and occlusion members 17, each planar spring 16 and occlusion member 17 may elastically deform to apply an appropriate occlusion pressure to an enclosed skin tag.

Alternatively, one occlusion member 17 and its connected planar springs 16 may be fixed or rigid, such that occlusion pressure is provided to a skin tag enclosed between the fixed or rigid occlusion member 17 and an opposing movable occlusion member 17 and planar springs 16. Moreover, the fixed or rigid occlusion member 17 and its connected planar springs 16 may form part of the base member 15.

Preferably, in a rest state of the pressure layer 14, a gap of approximately 20 mil (or 0.020 inches), or approximately 15 mil (or 0.015 inches), may be present between the occlusion members 17. More preferably, in a rest state of the pressure layer 14, there may be a zero gap between the occlusion members 17, i.e., the occlusion members 17 may contact each other in the rest state.

The pressure layer 14 may be made of a clear polyethylene terephthalate glycol (PETG) film, stainless steel, other suitable plastics and/or metals, or combinations thereof having a thickness of approximately 15 mil (or 0.015 inches). Alternatively, the thickness of the pressure layer 14 may vary from approximately 15 mil to approximately 30 mil (or 0.030 inches). In addition, in a top plan view, the pressure layer 14 may be approximately 0.8 inches long by approximately 0.8 inches wide. Further, in a top plan view, the width of the planar springs 16 may vary from approximately 0.04 inches to approximately 0.06 inches. The above dimensions of the pressure layer 14 represent one non-limiting embodiment of a device 10 according to the present invention. Other ranges of dimensions are possible, depending on the desired occlusion pressure and the size of the enclosed skin tag.

As shown in FIG. 3, the top layer 11 is configured to select, center, and align an enclosed skin tag. In addition, the top layer 11 may be formed in a shape substantially similar to that of the base member 15 of the pressure layer 14, and the top layer 11 includes a hole having specified diameter and specified location.

As set forth above with reference to FIG. 1, skin tags come in many different shapes and sizes. Users of prior art “self-treatment” devices have little to no guidance with respect to determining whether a skin tag is appropriate for treatment using a particular “self-treatment” device. As a result, users may select skin tags that are too large for effective treatment by a particular device. The consequences of such inappropriate selection of skin tags may include bleeding, blistering, scarring, infection, ineffective treatment, and others. The device 10 according to the present invention addresses this problem of inappropriate selection of skin tags. In particular, the top layer 11 of the device 10 is configured to select an appropriate skin tag for treatment. The top layer 11 includes a hole 12 of specified diameter that allows a skin tag having a diameter smaller than that of the hole 12 to pass through, whereas a skin tag having a diameter larger than that of the hole 12 is not allowed to pass through. Accordingly, only skin tags of appropriate size, i.e., diameter, may be enclosed in a top layer 11 of a device 10 having a hole 12 of particular diameter. For larger skin tags, a modified device 10 may be provided having a hole 12 of larger diameter and having the capability to provide different, e.g., higher, occlusion pressures appropriate for the larger skin tags. Thus, a specified diameter of the hole 12 of top layer 11 facilitates the appropriate selection of skin tags for treatment by users with little or no medical background. Appropriate selection of skin tags for use of the device increases the degree of effectiveness of this improved design over prior art designs.

In addition, users of prior art “self-treatment” devices have little to no guidance with respect to proper orientation and placement of the device relative to an enclosed skin tag. As a result, although users may have seemingly successfully applied a “self-treatment” device to a skin tag, treatment may be ineffective due to improper placement that results, for example, in ineffective levels of occlusion pressure due to shifting of the occlusion members out of plane. However, users have no way of knowing whether appropriate occlusion pressure is being applied to a skin tag, unless and until the skin tag is successfully removed. In all instances other than successful skin tag removal, users may determine that the device itself is defective, when in fact, proper placement would have led to successful treatment of the skin tag. Additional consequences of improper placement of the “self-treatment” device may include prolonged treatment, bleeding, blistering, scarring, infection, and others. The device 10 according to the present invention addresses this problem of improper placement of the device 10. Appropriate placement of skin tags increases the degree of effectiveness of this improved design over prior art designs. In particular, the top layer 11 of the device 10 may be configured to center a skin tag in the device 10 for treatment. The top layer 11 includes a hole 12 at a specified location, for example, in the center of the device 10, thereby centering the enclosed skin tag relative to the pressure layer 14, in particular, relative to the occlusion members 17 and the planar springs 16. Thus, appropriate occlusion pressure will be applied to the enclosed skin tag since proper orientation of the device relative to the skin tag is assured. Accordingly, a specified location of the hole 12 of top layer 11 facilitates proper placement of an enclosed skin tag in order to be able to provide appropriate occlusion pressure by the occlusion members 17 and the planar springs 16 for treatment by users with little or no medical background.

Further, prior art “self-treatment” devices suffer from difficulties of maintaining proper alignment. In this regard, alignment refers both to proper alignment of the occlusion members relative to each other to provide occlusion pressure, and proper alignment of the skin tag relative to the occlusion members to maintain occlusion pressure at the base of the skin tag. When occlusion members fail to maintain proper alignment with each other, appropriate occlusion pressure is no longer assured, and blood flow to the enclosed skin tag will not be occluded. Similarly, when an enclosed skin tag is not properly aligned with the occlusion members, the skin tag itself causes the occlusion members to come out of alignment, thereby preventing occlusion of blood flow to the enclosed skin tag. Consequences of improper alignment of the “self-treatment” device and skin tag may include prolonged treatment, bleeding, blistering, scarring, infection, and others. The device 10 according to the present invention addresses this problem of improper alignment of the device 10 and the skin tag. In particular, the top layer 11 of the device 10 is configured to maintain proper alignment of the planar springs 16 and the occlusion members 17 of the device 10. The top layer 11 is adhered to an upper side of the pressure layer 14, and covers at least a portion of the planar springs 16. Alternatively, the top layer 11 may cover at least a portion of the occlusion members 17 and the planar springs 16. Thus, the top layer 11 prevents movement of the planar springs 16 and the occlusion members 17 out of plane from each other, thereby maintaining proper alignment and occlusion pressure. Further, the top layer 11 includes a hole 12 configured to receive the enclosed skin tag, such that the enclosed skin tag remains perpendicular to the plane of the device 10 and blood flow to the enclosed skin tag is occluded. Moreover, even if the enclosed skin tag is subsequently bent or folded down by a cover strip, the top layer 11 prevents the skin tag from forcing the occlusion members 17 and planar springs 16 out of alignment from each other. Accordingly, the top layer 11 in combination with the hole 12 of the top layer 11 facilitates maintaining the planar alignment of the occlusion members 17 and the planar springs 16, and the perpendicular alignment of the skin tag relative to the top layer 11, to ensure proper occlusion of blood flow to the skin tag for treatment by users with little or no medical background. Appropriate alignment of the occlusion members increases the degree of effectiveness of this improved design over prior art designs.

Accordingly, the combination of having a top layer 11, a hole 12 of the top layer 11, specified diameter of the hole 12, and specified location of the hole 12 addresses multiple problems of the prior art “self-treatment” devices by facilitating appropriate selection of skin tags for treatment, proper orientation and placement of the device relative to the skin tag, and proper alignment of the device itself and of the skin tag relative to the device in a simple and industrially applicable way. In this manner, users with little or no medical background can reliably and effectively use the device according to the present invention with fewer potential modes of failure and adverse consequences of prior art designs.

Furthermore, the top layer 11 may also protect the pressure layer 14 from scuffing, unintentionally catching on clothing or other items, or otherwise being affected by external forces. Accordingly, the top layer 11 may protect the pressure layer 14, including the planar springs 16 and occlusion members 17, from being damaged.

The top layer 11 may be made of a clear polyethylene terephthalate glycol (PETG) film having a thickness of approximately 7 mil (or 0.007 inches). In addition, in a top plan view, the top layer 11 may be approximately 0.8 inches long by approximately 0.8 inches wide. Further, the hole 12 of the top layer 11 may have a diameter of approximately 0.1575 inches. The above dimensions of the top layer 11 represent one non-limiting embodiment of a device 10 according to the present invention. Other ranges of dimensions are possible, depending on the desired occlusion pressure and the size of the enclosed skin tag.

As shown in FIG. 4, the intermediate adhesive layer 13 may be situated between the pressure layer 14 and the top layer 11. The intermediate adhesive layer 13 may be formed in a shape substantially similar to that of the base member 15 of the pressure layer 14. For example, the intermediate adhesive layer 13 may preferably be shaped to cover only an upper side of the base member 15 of the pressure layer 14. Accordingly, when the pressure layer 14 and the top layer 11 are adhered together via the intermediate adhesive layer 13, the planar springs 16 and the occlusion members 17 of the pressure layer 14 may be free to move relative to the top layer 11. Alternatively, the intermediate adhesive layer 13 may cover more or less area than that of the base member 15. For example, the intermediate adhesive layer 13 may cover only a portion of the upper side of the base member 15, as opposed to the entire upper side of the bas member 15.

The intermediate adhesive layer 13 may be made of an acrylic pressure sensitive adhesive (PSA) having a thickness of approximately 3 mil (or 0.003 inches). In addition, in a top plan view, the intermediate adhesive layer 13 may be approximately 0.8 inches long by approximately 0.8 inches wide. Further, the PSA of the intermediate adhesive layer 13 may be any adhesive suitable for adhering the top layer 11 and the pressure layer 14 together in order to maintain proper function of the device 10. The above dimensions of the intermediate adhesive layer 13 represent one non-limiting embodiment of a device 10 according to the present invention. Other ranges of dimensions are possible, depending on the desired occlusion pressure and the size of the enclosed skin tag.

As shown in FIG. 6, the base adhesive layer 19 may be situated between the pressure layer 14 and the skin area. The base adhesive layer 19 may be formed in a shape substantially similar to that of the pressure layer 14. For example, the base adhesive layer 19 may preferably be shaped to cover the undersides of the base member 15, the planar springs 16, and the occlusion members 17 of the pressure layer 14. Accordingly, when the pressure layer 14 is adhered to the skin area via the base adhesive layer 19, the planar springs 16 and the occlusion members 17 of the pressure layer 14 may be fixed in order to maintain the occlusion pressure applied to an enclosed skin tag. Alternatively, the base adhesive layer 19 may cover more or less area than that of the pressure layer 14. For example, the base adhesive layer 19 may cover only portions of the undersides of one or more of the base member 15, the planar springs 16, and the occlusion members 17.

The base adhesive layer 19 may be made of an acrylic pressure sensitive adhesive (PSA) having a thickness of approximately 3 mil (or 0.003 inches). In addition, in a top plan view, the base adhesive layer 19 may be approximately 0.8 inches long by approximately 0.8 inches wide. Further, the PSA of the base adhesive layer 19 may be any adhesive suitable for adhering the pressure layer 14 to the skin area in order to maintain proper function of the device 10. Prior to application to the skin area, e.g., during fabrication, shipment and/or storage, the base adhesive layer 19 may include a 96 lb. lay flat release liner, such as for example, Loparex 15082 or equivalents. The release liner may be removed in order to expose the base adhesive layer 19 for application to the skin area. The above dimensions of the base adhesive layer 19 represent one non-limiting embodiment of a device 10 according to the present invention. Other ranges of dimensions are possible, depending on the desired occlusion pressure and the size of the enclosed skin tag.

The device 10 may generally be designed for skin tags having a diameter of 3 mm or smaller. Thus, in one non-limiting embodiment, the pressure layer 14, and in particular, the planar springs 16 and occlusion members 17, may be designed to apply occlusion pressure appropriate for skin tags of 3 mm diameter or smaller. In addition, the top layer 11, and in particular, the hole 12 of the top layer 11, may be designed to receive skin tags of 3 mm diameter or smaller.

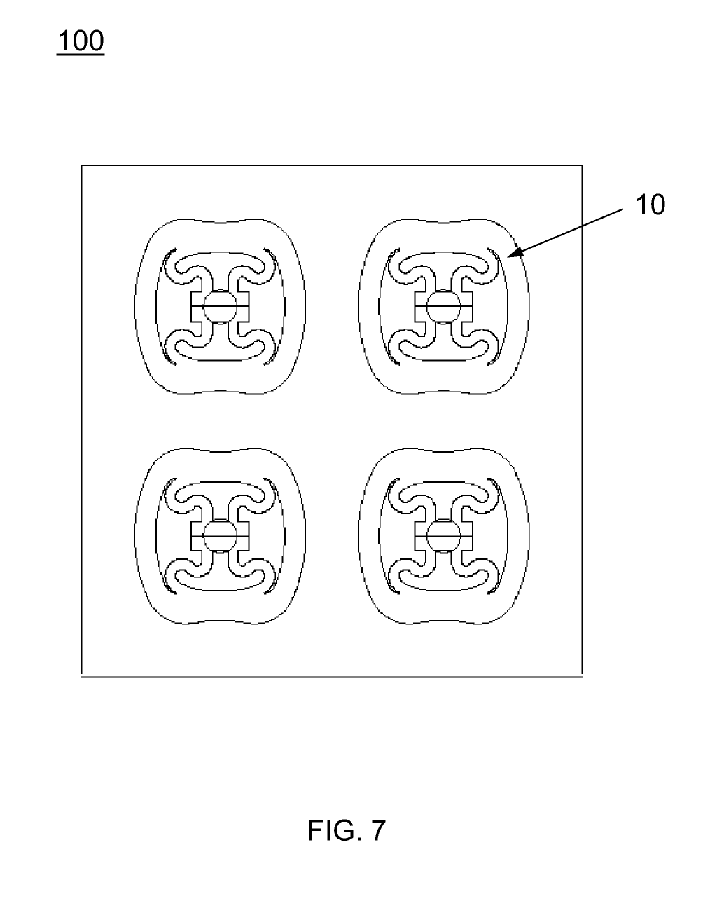

FIG. 7 illustrates a top plan view of a card 100 including a plurality of an exemplary embodiment of a device 10 according to the present invention. Multiple devices 10 may be formed together on a card 100 including each of the layers of the device 10, i.e., the top layer 11, the intermediate adhesive layer 13, the pressure layer 14, and the base adhesive layer 19. Then, individual devices 10 may be removed from the card 100 for use at the appropriate time.

The card 100 may vary in size and may include a variable number of devices 10. For example, as shown in FIG. 7, the card 100 may include four devices 10 that are spaced apart from each other by approximately 0.25 inches. Alternatively, the card 100 may include fewer or more devices 10 with different spacing between individual devices 10.

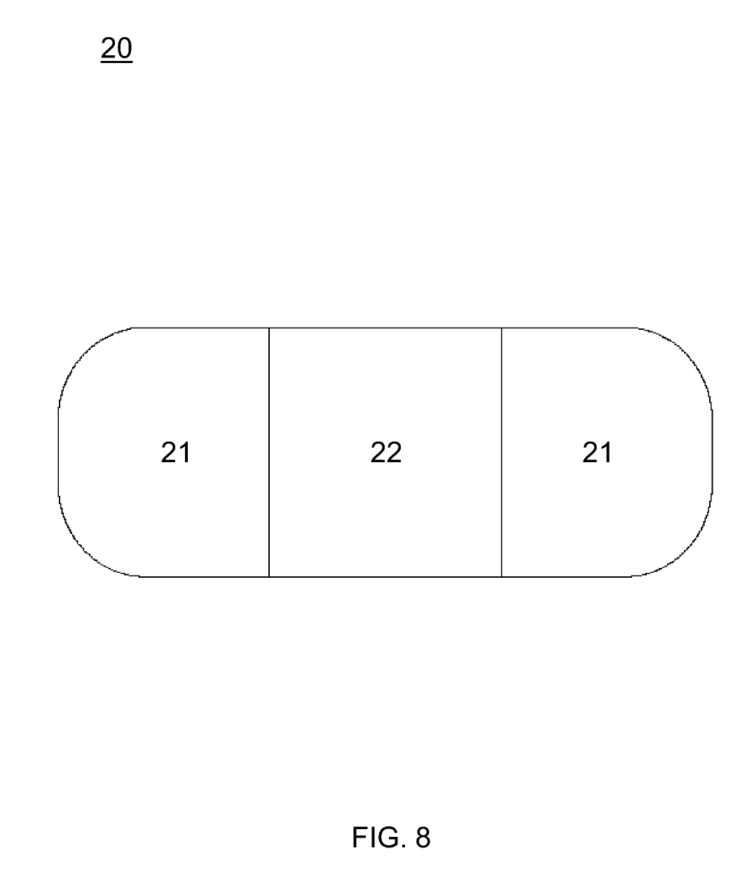

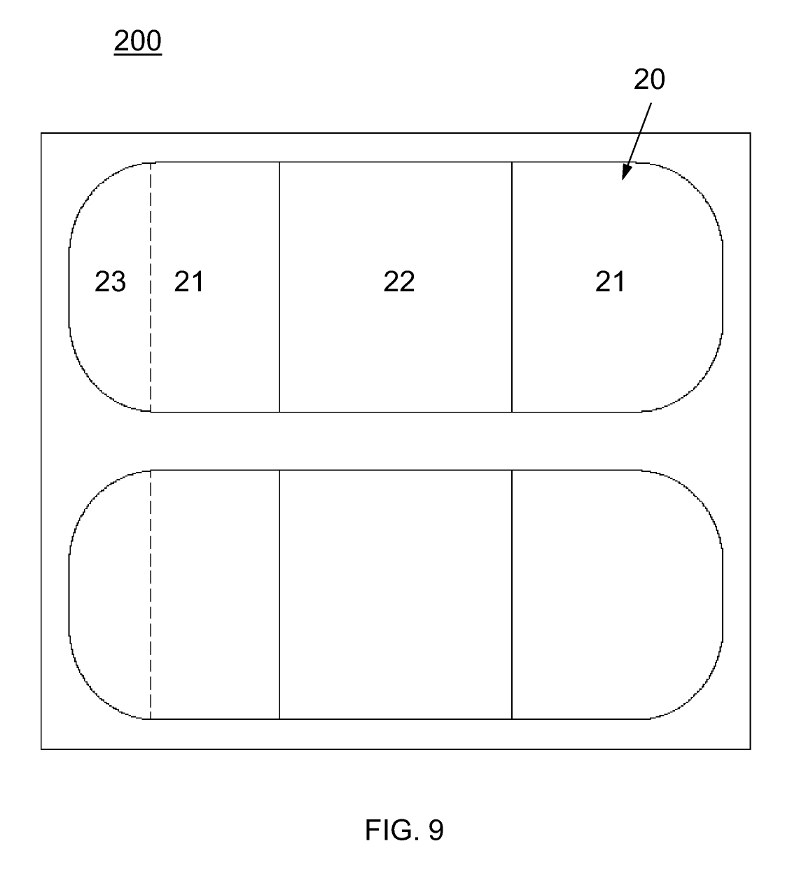

FIG. 8 illustrates a top plan view of a cover strip 20 for an exemplary embodiment of a device 10 according to the present invention. The cover strip 20 may include a clear cover film 21, a tan cover film 22, adhesive on an underside (not shown), and a paper tab 23 (shown in FIG. 9).

The clear cover film 21 may span the entire length and width of the cover strip 20. The clear cover film 21 may also include an adhesive on its underside.

The clear cover film 21 may be made of a clear, perforated vinyl film coated with an acrylic PSA, such as for example, Avery-Dennison MED 5511-28350-50. In addition, in a top plan view, the clear cover film 21 may be approximately 1.0 inches wide and approximately 3.0 inches long. Moreover, the corners of the clear cover film 21 may include a radius of approximately 0.4 inches. Further, the PSA of the clear cover film 21 may be any adhesive suitable for adhering to the device 10 and/or the skin area. The above dimensions of the clear cover film 21 represent one non-limiting embodiment of a cover strip 20 for a device 10 according to the present invention. Other ranges of dimensions are possible, depending on the sizes of the device 10 and the enclosed skin tag.

The tan cover film 22 may be situated only in a central area of the clear cover film 21. The tan cover film 22 may be adhered to an underside of the clear cover film 21. The tan cover film 22 may also include an adhesive on its underside. Preferably, however, the tan cover film 22 does not include an adhesive on its underside.

The tan cover film 22 may be made of a tan vinyl film coated with an acrylic PSA, such as for example, Avery-Dennison MED 5512-28354-50, although the tan cover film 22 preferably does not include PSA on its underside. In addition, in a top plan view, the tan cover film 22 may be approximately 1.0 inches wide and approximately 1.0 inches long. The above dimensions of the tan cover film 22 represent one non-limiting embodiment of a cover strip 20 for a device 10 according to the present invention. Other ranges of dimensions are possible, depending on the sizes of the device 10 and the enclosed skin tag.

A paper tab 23 may be situated at one end of the clear cover film 21 in order to facilitate application of the cover strip 20 by removal of the paper tab 23. Alternatively, paper tabs 23 may be situated at opposing ends of the clear cover film 21.

The paper tab 23 may be made of a 42 lb. release liner, such as for example, Master Roll 23984-50. In addition, in a top plan view, the paper tab 23 may be approximately 1.0 inches wide and approximately 0.375 inches long. The above dimensions of the paper tab 23 represent one non-limiting embodiment of a cover strip 20 for a device 10 according to the present invention. Other ranges of dimensions are possible, depending on the sizes of the device 10 and the enclosed skin tag.

Prior to application to the skin area, e.g., during fabrication, shipment and/or storage, the cover strip 20 may include a 96 lb. lay flat release liner, such as for example, Loparex 15082 or equivalents. The release liner may be removed in order to expose the PSA of the clear cover film 21 for application to the skin area.

The cover strip 20 may be applied on top of the device 10 in order to cover the enclosed skin tag. The cover strip 20 may adhere to the skin area, the device 10, and/or the enclosed skin tag. Preferably, however, the cover strip 20 does not adhere to the enclosed skin tag because preferably, no adhesive is present on the underside of the tan cover film 22. Accordingly, the cover strip 20 may be removed from the skin area and/or the device 10 without having to unaffix the enclosed skin tag from adhesive of the cover strip 20.

FIG. 9 illustrates a top plan view of a card 200 including a plurality of cover strips 20 for an exemplary embodiment of a device 10 according to the present invention. Multiple cover strips 20 may be formed together on a card 200 including each of the layers of the cover strip 20, i.e., the clear cover film 21, the tan cover film 22, the paper tabs 23, and the adhesive. Then, individual cover strips 20 may be removed from the card 200 for use at the appropriate time.

The card 200 may vary in size and may include a variable number of cover strips 20. For example, as shown in FIG. 9, the card 200 may include two cover strips 20 that are spaced apart from each other by approximately 0.25 inches. Alternatively, the card 200 may include fewer or more cover strips 20 with different spacing between individual cover strips 20.

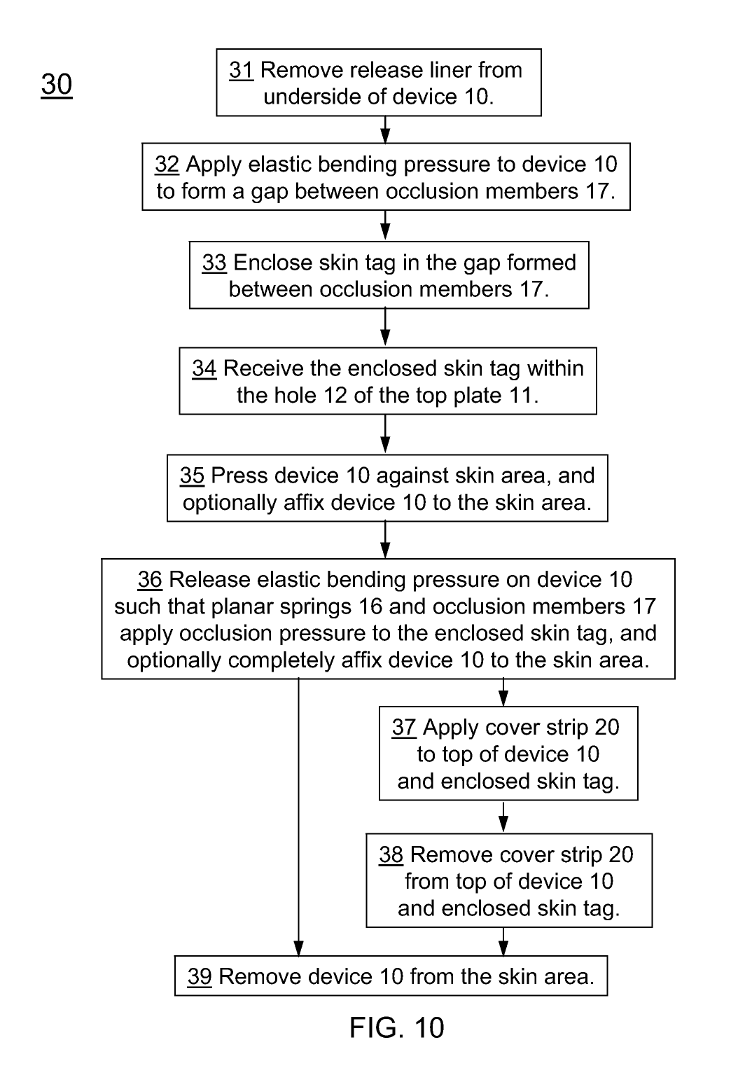

FIG. 10 illustrates a schematic flow diagram of a method 30 of using a device 10 according to the present invention.

After a skin tag is identified for occlusion, a device 10 according to the present invention may be chosen for application to the identified skin tag.

At step 31, the user may remove a release liner from an underside of the device 10, thereby exposing the base adhesive layer 19 of the device 10.

At step 32, the user may grasp the device 10 and apply pressure to the device 10, e.g., by holding the device 10 between a thumb and a finger, for example, with one hand, using the finger notches 18 of the device 10, such that the device 10 elastically bends away from the grasping thumb and finger and toward the identified skin tag. The elastic bending of the device 10 may allow the formation of a gap between the occlusion members 17 of the device 10.

At step 33, the user, while maintaining the elastically bent configuration of the device 10, may maneuver the device 10 such that the identified skin tag is enclosed within the gap formed between the occlusion members 17.

At step 34, the user, while still maintaining the elastically bent configuration of the device 10, may maneuver the device 10 such that the enclosed skin tag is received within the hole 12 of the top layer 11, thereby selecting, centering, and aligning the enclosed skin tag with the device 10.

At step 35, the user may press the device 10 against the skin area, and may optionally begin to affix the device 10 to the skin area 2 via the base adhesive layer 19.

Then, at step 36, the user may release the pressure on the device 10, such that the device 10 returns to its original, e.g., flat, configuration. Upon releasing the manual pressure on the device 10, e.g., between the user's thumb and finger, the planar springs 16 and the occlusion members 17 of the device 10 may apply occlusion force sufficient to occlude blood flow to the enclosed skin tag, preferably to a base of the enclosed skin tag. The user may then also completely affix the device 10 to the skin area via the base adhesive layer 19.

At step 37, after the device 10 has been affixed to the skin area, and the planar springs 16 and the occlusion members 17 are configured to occlude blood flow to the enclosed skin tag, a cover strip 20 may be applied to the top of the device 10, the skin area, and/or the enclosed skin tag.

At step 38, the cover strip 20 may be removed from the top of the device 10, the skin area, and/or the enclosed skin tag. Depending on the progress of the occlusion of blood flow to the skin tag, steps 37 and 38 may be repeated as necessary to protect the device 10 and/or the skin tag from external forces and other environmental influences until the treatment of the enclosed skin tag is complete.

Alternatively, the user may skip both of steps 37 and 38 and leave the device 10 uncovered throughout the duration of the treatment. In such a case, the user may proceed directly to step 39 from step 36 when treatment of the enclosed skin tag is complete.

At step 39, after the skin tag has been fully occluded, the device 10 may be removed from the skin area. If the skin tag is ready to be removed from the skin area, the skin tag may be removed at the same time as removal of the device 10. However, if the skin tag has been sufficiently occluded but is not yet ready to be removed from the skin area, the device 10 may be removed from the skin area first, and the skin tag may be removed from the skin area after some time without further application of the device 10. That is, although blood flow to the skin tag has been sufficiently occluded, the skin tag may remain attached to the skin area for some time after removal of the device 10, until the skin tag is ready to fall off the skin area without application of additional trauma.

In an exemplary embodiment of the device 10 according to the present invention, the desired applied force to be applied to an enclosed skin tag is 150 grams of force at a compression distance of 0.3 mm. In order to achieve the desired applied force, experiments were performed on sample devices having various thicknesses of the pressure layer 14, widths (in a top plan view) of the planar springs 16, and overall sizes (in a top plan view) of the device 10.

The thickness of the pressure layer 14 was varied between approximately 0.015 inches to approximately 0.030 inches. The width of the planar springs 16 was varied between approximately 0.040 inches to approximately 0.060 inches. The overall size of the device 10 was varied between approximately 0.8 inches×0.8 inches to approximately 1.2 inches×1.2 inches.

The sample devices were manufactured by laser cutting to the various dimensions using, for example, an Epilog Model Mini 24 Laser Cutter. The kerf thickness for the laser cutting of the sample devices was approximately 0.02 inches.

Compression testing was performed on the sample devices at compression distances of approximately 0.3 mm and approximately 1.3 mm. Based on the results of the compression testing at approximately 0.3 mm, it was determined that the thickness of the pressure layer 14 has relatively little effect on the compressive force measurements at 0.3 mm compression, and the error associated with any correlation between the overall size of the sample devices and the compressive force measurements was too large. However, the width of the planar springs 16 provides an approximately linear correlation to the compressive force measurements.

Further, based on the results of the compression testing at approximately 1.3 mm, yielding of the pressure layer 14 and/or planar springs 16 due to plastic deformation of the material was not a major concern. In other words, for the various widths of the planar springs 16 having a pressure layer 14 thickness of approximately 0.015 inches, neither the pressure layer 14 nor the planar springs 16 plastically deformed under the compression forces at a compression distance of 1.3 mm.

Accordingly, based on the experimental testing of the sample devices, it was determined that a non-limiting embodiment of the device 10 having a pressure layer 14 with a thickness of approximately 0.015 inches, a width of planar springs 16 of approximately 0.040 inches, and an overall size of approximately 0.8 inches by 0.8 inches is sufficient to achieve the desired applied force of 150 grams at a compression distance of 0.3 mm. Moreover, the width of the planar springs 16 exhibits a linear correlation to compression force such that the width may be varied in order to correspondingly vary the compression force achievable by a particular device. The above dimensions of the device 10 represent one non-limiting embodiment of a device 10 according to the present invention for a specified desired applied force of 150 grams at a compression distance of 0.3 mm. Other ranges of dimensions are possible, depending on the desired applied force and compression distance.

The foregoing description discloses only non-limiting embodiments of the present invention. Modification of the above-disclosed exemplary devices, and a method of using the same, which fall within the scope of the invention, will be readily apparent to those of ordinary skill in the art.

Accordingly, while the present invention has been disclosed in connection with the above non-limiting embodiments, it should be understood that other embodiments may fall within the spirit and scope of the invention, as defined by the following claims.

What is claimed is:

1. A device for occlusion of a skin tag projecting from a skin area, comprising:
a pressure layer having an upper side, an underside, and configured to enclose the skin tag and occlude blood flow to the enclosed skin tag, the pressure layer comprising:
a base member having an upper side, an underside, and configured to enclose the skin tag;
at least one first planar spring connected to a first side of the base member at one end of the first planar spring; and
a first occlusion member connected to an other end of the first planar spring for applying an occlusion force to the enclosed skin tag; and
a top layer attached to the upper side of the pressure layer, the top layer covering at least a portion of the first planar spring, and the top layer having a hole configured to receive the enclosed skin tag.

2. The device according to claim 1, wherein the pressure layer further comprises:
at least one second planar spring connected to a second side of the base member at one end of the second planar spring; and
a second occlusion member connected to an other end of the second planar spring for applying the occlusion force to the enclosed skin tag.

3. The device according to claim 1, wherein the underside of the pressure layer includes an adhesive for fixing the pressure layer to the skin area.

4. The device according to claim 1, wherein the upper side of the pressure layer includes an adhesive for fixing the top layer to the pressure layer.

5. The device according to claim 2, wherein the other end of the first planar spring faces the other end of the second planar spring.

6. The device according to claim 2, wherein the first planar spring and the second planar spring are formed substantially in the shape of a “S”.

7. The device according to claim 1, wherein a pair of first planar springs is connected to the first occlusion member.

8. The device according to claim 2, wherein a pair of second planar springs is connected to the second occlusion member.

9. The device according to claim 2, wherein the first planar spring and the second planar spring are made of at least one of plastic, metal, clear polyethylene terephthalate glycol (PETG) film, and etched stainless steel.

10. The device according to claim 2, wherein the first occlusion member faces the second occlusion member for jointly applying the occlusion force to the enclosed skin tag.

11. The device according to claim 2, wherein in a rest state, a gap is present between the first occlusion member and the second occlusion member.

12. The device according to claim 11, wherein the gap is between approximately zero and approximately 20 mil.

13. The device according to claim 1, wherein the device is one of flat and disc-shaped.

14. The device according to claim 1, wherein the device is substantially square with rounded edges.

15. The device according to claim 1, wherein the top layer is configured to at least one of select, center, and align the enclosed skin tag for occlusion.

16. The device according to claim 1, wherein the first side and the second side of the base member of the pressure layer are configured to provide finger notches.

17. The device according to claim 1, further comprising:
a cover strip configured to cover at least one of the pressure layer, the top layer, and the enclosed skin tag.

18. The device according to claim 17, wherein an underside of the cover strip includes an adhesive configured to adhere to at least one of the pressure layer, the top layer, the enclosed skin tag, and the skin area.

19. The device according to claim 18, wherein the underside of the cover strip includes the adhesive along an entire length of the cover strip.

20. The device according to claim 18, wherein the underside of the cover strip does not include adhesive in a central region of the cover strip configured to cover the enclosed skin tag.

21. A method of using a device for occlusion of a skin tag projecting from a skin area, the device comprising a pressure layer having an upper side, an underside, and configured to enclose the skin tag and occlude blood flow to the enclosed skin tag, the pressure layer comprising a base member having an upper side, an underside, and configured to enclose the skin tag, at least one first planar spring connected to a first side of the base member at one end of the first planar spring, and a first occlusion member connected to an other end of the first planar spring; and a top layer attached to the upper side of the pressure layer, the top layer covering at least a portion of the first planar spring, and the top layer having a hole, the method comprising:
receiving the enclosed skin tag within the hole of the top layer.

22. The method according to claim 21, wherein the pressure layer further comprises at least one second planar spring connected to a second side of the base member at one end of the second planar spring, and a second occlusion member connected to an other end of the second planar spring, the method further comprising:
applying compression to the device, whereby a gap is formed between the first and second occlusion members;
enclosing the skin tag within the gap formed between the first and second occlusion members; and
releasing the compression on the device, whereby occlusion force is applied to the enclosed skin tag via the first and second occlusion members and the first and second planar springs.

23. The method according to claim 22, wherein the compression is manually applied by a user's thumb and finger via finger notches provided at the first side and the second side of the base member of the pressure layer.

24. The method according to claim 22, wherein the device is applied with one hand of a user.

25. The method according to claim 21, further comprising:
adhering the pressure layer to the skin area via an adhesive.

26. The method according to claim 21, wherein the top layer at least one of selects, centers, and aligns the enclosed skin tag for occlusion.

27. The method according to claim 21, further comprising:
covering at least one of the pressure layer, the top layer, and the enclosed skin tag with a cover strip.

28. The method according to claim 27, further comprising:
adhering the cover strip to at least one of the pressure layer, the top layer, the enclosed skin tag, and the skin area via an adhesive.

29. The device according to claim 27, wherein the covering includes covering the enclosed skin tag with the cover strip without adhering the cover strip to the enclosed skin tag.
